(19) United States
(12) Patent Application Publication
(10) Pub. No.: US 2019/0374370 A1
(43) Pub. Date: Dec. 12, 2019

(54) SHEAR FORCE NEGATING PLASTER

(71) Applicants: Leonard Vincent Waters, Wirral (GB); Karl Ormond, Wirral (GB)

(72) Inventors: Leonard Vincent Waters, Wirral (GB); Karl Ormond, Wirral (GB)

(21) Appl. No.: 16/463,217

(22) PCT Filed: Dec. 2, 2016
(86) PCT No.: PCT/GB2016/053799

§ 371 Date: May 22, 2019

(51) Int. Cl. A61F 5/34; A61F 13/06

(52) U.S. Cl. CPC A61F 5/34; A61F 2013/00404; A61F 13/069

(57) ABSTRACT

A shear force negating plaster (10) in which the top layer of material consists of a Baltex spacer material (14), which because of its construction allows a degree of lateral movement so as to compliment the lateral movement of the two flexible planar circular plastic inserts (16, 20).

This invention relates to a plaster, a method of using a plaster, a method of preventing a skin blister and/or ulcer and an item of sportswear comprising a plaster. In particular, the present invention relates to a plaster for preventing skin blistering, more particularly, skin blistering that can occur to areas of the skin primarily in relation to the foot or areas of the foot that are subject to lateral rubbing of the skin which can cause separation and fluid build-up resulting in blistering.

Due to almost all sports or leisure activities that are carried out on hard surfaces where because of lateral movement combined with forced contact to the ground, heat and moisture and the lateral rubbing affect can quickly result in blistering that not only impedes performance but can also be very painful for a subject during the healing process.

This present invention may also be used as a preventative treatment for diabetics who because of their illness suffer badly from sores and blisters to their feet that can deteriorate to the point where amputation may be necessary.

The present invention may address and/or alleviate any one or more of the problems associated with the prior art.

There is provided a plaster, a method of using a plaster, a method of preventing a skin blister and an item of sportswear as defined in any one of the accompanying claims.

In an aspect of the present invention, there is provided a shear force negating plaster comprising a base having an adhesive disposed on its lower surface and intended to contact and adhere to a substrate, an upper layer connected to the base and forming a chamber therebetween, wherein the chamber houses at least two moveable members and a lubricant for facilitating movement of the moveable members over one another.

The upper layer may comprise a cushioned material, such as a Baltex (available from Baltex Ltd) spacer material or other compatible material upper, a woven fabric or similar base layer. The material may comprise polyester. The material advantageously comprises polyester and cotton and optionally, one or more synthetic yarns, for example, Lycra® and/or Coolmax® (both available from Invista Sarl).

Alternatively, the base may comprise a cushioned material as described herein. In an embodiment, both the base and upper layer may comprise a cushioned material as described herein.

The moveable members may have a round shape or other shape, such as an oblong or square shape. The moveable members may be flexible plastic inserts. The moveable members may be planar. The moveable members are advantageously disc shaped.

The moveable members may be flexible.

Advantageously, there are two moveable members.

The chamber may contain a gel or similar type of lubricant which is intended to reduce the friction between the moveable members.

The gel or similar type of lubricant may be layered in between the two flexible plastic inserts, and then placed in between the Baltex spacer material or other compatible material and laminated into a complete unit.

The substrate may comprise an item of sportswear, such as an item of footwear, helmet, shirt, shorts, trousers, socks, protective wear or the like.

There is also provided a shear force negating plaster comprises of a Baltex spacer material or other compatible material upper, and a woven fabric or similar base layer of which two round or other shape possibly even oblong or square flexible plastic inserts with a gel or similar type of lubricant which is layered in between the two flexible plastic inserts, and then placed in between the Baltex spacer material or other compatible material and laminated into a complete unit.

Known plasters tend to focus on reducing the possibility of developing blisters by adhering to foot where blistering is likely to occur in hot and moist conditions which tends to result in the plaster coming away from the venerable area leaving the area exposed to blistering.

The shear force negating plaster of the present invention may adhere to the inside of the footwear directly to the area the subject feels they are likely to be exposed to lateral rubbing which will cause the skin blistering to develop.

The shear force negating plaster of the present invention may also be versatile enough so as to be incorporated into other products where skin blistering is a common problem, for example gloves, socks or different types of joint protection.

The shear force negating plaster may be substantially circular in shape but any other suitable shape is encompassed by the present invention, including square, rectangular, ovoid, v-shaped, y-shaped and the like. The plaster of the present invention may have a shape that can vary in size and form in relation to where it is positioned on a subject's body or a garment, piece of footwear, glove, helmet, and other such accessories.

For example, if the plaster of the present invention were positioned in the toe area or the front of an item of footwear, it might be appropriate to use an oblong shaped plaster with oblong flexible plastic inserts. In contrast, for example, if the plaster were to be positioned into a curved area on the side of the footwear an oval shaped plaster may be more appropriate with oval flexible plastic inserts.

The top layer of the plaster may comprise any type of material that would allow a degree of lateral movement as to allow the flexible plastic inserts with a gel or similar type of lubricant incorporated between the two flexible plastic inserts to slide laterally, so as to negate any rub transferring from the footwear to the skin of a subject which can cause blistering thereof.

The preferred top layer of material would be a readily available Baltex spacer material which is 2.5 mm to 3 mm thick but is flexible in its lateral ability which contributes to its maximum effectiveness of the two flexible plastic inserts incorporating a layer of gel or similar type lubricant in between the two flexible plastic inserts.

The base layer can be a standard plaster type material or similar type material that would allow enough rigidity so as to give the plaster the ability to adhere to the inside of the footwear but not too thin a material that would allow the plaster to be susceptible to being forced away from its adhered position due to rubbing against it causing roll up of its edges.

The outside circumference of the plaster would be heat sealed to a width of around 5 mm so as to seal the flexible plastics inserts in place so as to allow lateral movement without cutting and separating the laminated top and bottom layers of the material used to construct the shear force negating plaster.

This would also act as a tapering off the edge of the plaster so as to seat firmly against the material of the footwear so as not to be susceptible to peeling away because of rubbing against it by the foot.

The pressure sensitive adhesive base layer applied to the base of the lower later of material could vary in strength depending on its various applicable needs, it may not be necessary if the plaster was to be incorporated into a glove or a sock but depending on the type of footwear it would probably differ in strength if used in a running shoe as opposed to be used in a leather type football boot due to its effectiveness in negating lateral shear force in sports such as running, football, rugby, tennis, climbing and long distance walking it would have a variety of materials to be considered to which it can be applied.

There is also provided a patch for contacting a portion of a subject, said patch being a laminate comprising a base layer having an adhesive capable of adhering to a substrate and an upper layer for contacting a portion of a subject in use, the patch having at least two moveable members disposed in a chamber between the base layer and upper layer and capable of moving within the chamber in response to movement of the portion of a subject in contact with the upper layer.

The moveable member may comprise a planar circular, ovoid, square, rectangular shape.

The at least one moveable member may be disposed within a chamber disposed between the backing layer and upper layer.

The chamber may contain a fluid. The fluid may comprise a lubricant to reduce friction between the moveable members and/or the inner surface of the base and upper layer.

The moveable member may comprises a circular, square or oblong body. The moveable member may be resiliently deformable.

The upper layer may comprise a Baltex spacer material as described hereinabove.

The base may comprise a woven material.

The substrate may be an item of footwear.

There is also provided an item of footwear comprising a plaster as described hereinabove. There is also provided a shear force negating plaster comprises of a Baltex spacer material or other compatible material upper, and a woven fabric or similar base layer of which two round or other shape possibly even oblong or square flexible plastic inserts with a gel or similar type of lubricant which is layered in between the two flexible plastic inserts, and then placed in between the Baltex spacer material or other compatible material and laminated into a complete unit.

The shear force negating effect may be provided by two shaped plastic inserts with a layer of gel or similar lubricant that is heat sealed into place within a circumference of 5 mm space to allow movement in any direction.

The two shaped flexible plastic inserts may have a layer of gel or similar lubricant in between would absorb any lateral movement caused by contact between a part of the footwear coming into contact with an area of skin and causing soreness or blistering.

The two plastic flexible inserts may have a layer of gel or similar lubricant in between may be sealed into place between a top layer of Baltex spacer material or similar compatible material and a woven fabric or similar type material base layer can be adhered in place because of its pressure sensitive adhesive base in any place were friction between a material and the skin can cause sores or blistering allowing maximum protection.

The adhesive disposed on the base may have a carrier sheet disposed thereon to prevent the plaster adhering to substrates until required, the carrier sheet being capable of being removed when required to expose the adhesive.

The invention will now be described, by way of example only, with reference to the accompanying drawings, in which:

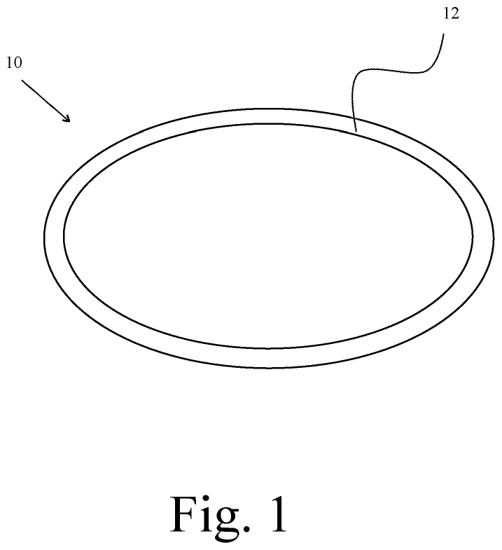

FIG. 1 shows a shear force negating plaster in accordance with the present invention; and

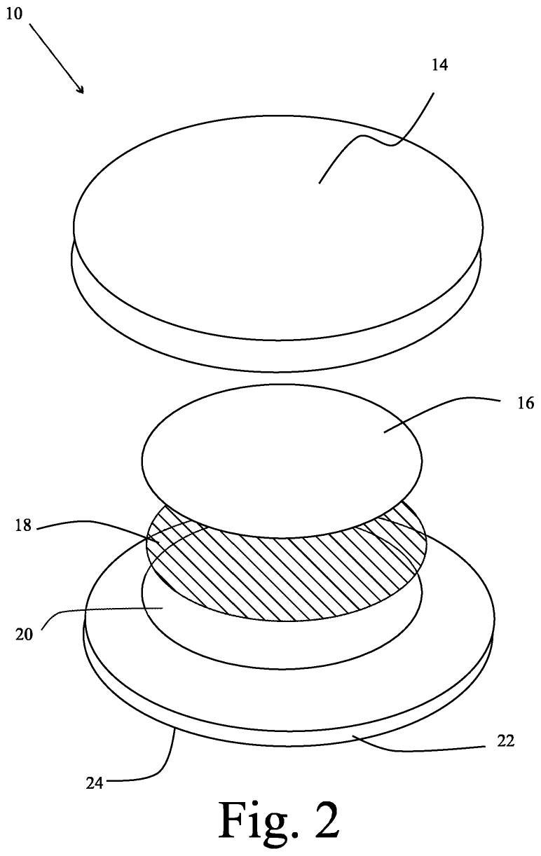

FIG. 2 shows an exploded view of the plaster of FIG. 1.

The composite makeup of the planar circular shear force negating plaster FIG. 1 shows the complete shear force negating plaster 10, laminated together with a heat sealed circumferential lip 12 of 5 mm or more depending on the size of the plaster so as to allow neat firm fixing of the plaster and its peripheral edge so as not to allow rolling up and pulling away.

FIG. 2 shows an exploded view of the shear force negating plaster 10 in which the top layer of material consists of a Baltex spacer material 14, which because of its construction allows a degree of lateral movement so as to compliment the lateral movement of the two flexible planar circular plastic inserts 16, 20, although other available materials can also be used so as to widen its variety of applications.

FIG. 2 also shows a layer of gel or similar type lubricant 18, in between the two flexible plastic inserts 16, 20, which should be just enough to allow free lateral movement but not too much so as to allow the plastic inserts to float father than slide.

The bottom layer of plaster, the base, 22 has a planar circular body of a pliable type material which should be stable enough so as to allow a firm adhesion to the inside of footwear, for example, so as to not allow the plaster to roll at the edges and dislodge. An adhesive is disposed on the lower surface 24 of the base 22. The base is coated with a pressure sensitive adhesive layer so as to adhere to whatever material the plaster would be applied to.

1. A shear force negating plaster comprising a Baltex spacer material or other compatible material upper, and a woven fabric or similar base layer of which two round or other shape possibly even oblong or square flexible plastic inserts with a gel or similar type of lubricant which is layered in between the two flexible plastic inserts, and then placed in between the Baltex spacer material or other compatible material.

2. The plaster of claim 1, wherein the upper and base are laminated into a complete unit.

3. The shear force negating plaster according to claim 1, in which the shear force negating effect is provided by two shaped plastic inserts with a layer of gel or similar lubricant that is heat sealed into place within a circumference of 5 mm space to allow movement in any direction.

4. The shear force negating plaster according to claim 1 in which the two shaped flexible plastic inserts with a layer of gel or similar lubricant in between absorb lateral movement caused by contact between a part of an item of footwear adjacent to an area of skin of a subject.

5. The shear force negating plaster according to claim 1 4 in which the two plastic flexible inserts with a layer of gel or similar lubricant in between can be sealed into place between a top layer of Baltex spacer material or similar compatible material and a woven fabric or similar type material base layer can be adhered in place because of its pressure sensitive adhesive base in any place were friction between a material and the skin can cause sores or blistering allowing maximum protection.

6. A method for preventing a skin blister on a subject comprising the use of a plaster as claimed in claim 1.

7. An item of sportswear comprising a plaster as claimed in claim 1.
